(19) United States
(12) Patent Application Publication
(10) Pub. No.: US 2019/0125312 A1
(43) Pub. Date: May 2, 2019

(54) ULTRASOUND IMAGING METHOD

(71) Applicant: General Electric Company, Schenectady, NY (US)

(72) Inventors: Zhiqiang Jiang, Wuxi (CN); Peter Lysyansky, Haifa (IL); Gang Liu, Wuxi (CN); Jiajiu Yang, Wuxi (CN); Lei Liu, Shanghai (CN)

(21) Appl. No.: 16/176,454

(22) Filed: Oct. 31, 2018

(30) Foreign Application Priority Data
Nov. 1, 2017 (CN) 201711058893.5

(51) Int. Cl. A61B 8/08; G06T 7/00; G06T 7/246; A61B 8/00; A61B 8/14

(52) U.S. Cl. CPC A61B 8/5276; G06T 7/0012; G06T 7/246; A61B 8/5246; A61B 8/463; A61B 8/0883; A61B 8/488; A61B 8/469; A61B 8/468; G06T 2207/30048; G06T 2207/10132; A61B 8/486; A61B 8/14

(57) ABSTRACT

The present invention provides an ultrasound imaging method, comprising: selecting an initial ultrasound image frame; marking a plurality of marking points on the initial ultrasound image frame; tracking updated positions of the marking points in subsequent ultrasound image frames; displaying updated marking points at the updated positions.

### FIELD OF INVENTION

The present invention relates to the field of medical imaging, particularly to an ultrasound imaging method.

### BACKGROUND OF THE INVENTION

The conventional ultrasound imaging system comprises an array of ultrasound sensor elements for transmitting ultrasound beams and receiving reflected ultrasound wave beams from an object that is being studied. By selecting a phase delay and an amplitude of applied voltage, each of transducer elements can be controlled to generate ultrasound waves, which are combined together to form net ultrasound waves travelling in a preferred vector direction and accumulating at a chosen point along the wave beams. A plurality of firing may be used to collect data representing the same anatomic information. Wave beam formation parameters for each firing may be changed so as to provide a change of focus length or otherwise change a content of the received data for each firing, for example, by transmitting continuous wave beams along the same route (a focus of each wave beam shifts with respect to a focus of the previous wave beam). By changing a phase rotation and amplitude of the input voltage supplied to the transducer element, the ultrasound wave beams may be moved to scan the object.

When the ultrasound data is collected and imaged, there are A-mode, B-mode and M-mode imaging methods in the existing technology. The A-mode (Amplitude Mode) ultrasound imaging belongs to an amplitude modulation imaging, which uses intensities of the ultrasound echo signals to adjust a baseline of a display to be high or low and displays in a one-dimensional waveform. The B-mode (Brightness Mode) ultrasound imaging is a brightness modulation imaging system, which uses the intensities of the ultrasound echo signals to adjust a brightness of the display, which displays thereon a two-dimensional section image, a horizontal direction and a vertical direction of the display representing a section width and an exploration depth respectively. The M-mode (Motion Mode) is the same as the A-mode in excitation of transducers and processing of echo signals, with their difference being that the M-mode adopts the B-mode method in outputting images, i.e., displaying a curve formed of swinging echoes by brightness. The M-mode is suitable for scanning a moving object (e.g., heart) to be imaged. Refer to FIG. 1B, in which the leftmost image illustrates a standard M-mode imaging for a heart valve, and a pulsation curve in the lower part of the image clearly shows a periodic motion of the heart valve.

In recent years, further improvements and developments have been made on the M-mode imaging method, including AMM (Anatomic M Mode) and TVM (Tissue Velocity Mode) methods, which have been widely applied to the ultrasound imaging system. The AMM may place a sampling line at random within a range of 360 degrees, thus obtaining an M-mode ultrasound image of any point and any angle; the TVM may display regions of a tissue with different velocities in different colors by using a Doppler effect, thus acquiring a motion velocity profile of the tissue intuitively. Due to the above advantages of the AMM and TVM, at present they are widely used in echocardiography to obtain a heart valve motion rhythm and a myocardium velocity profile, etc.

At present, the AMM and TVM imaging still have technical problems, which are mainly: “tissue lost” caused by the motion of the organ (mainly heart) and degradation/distortion of the ultrasound image caused by the tissue lost. Refer to FIG. 1A, in which the left image (at the time of T1) shows AMM imaging for the heart valve (the upper part) and TVM imaging for the myocardium (the lower part); In the imaging for the heart valve, the scanning line 11 passes through the mitral valve 12. In the imaging for the myocardium, the myocardium 13 is marked in dots and line segments along a periphery of the myocardium 13. Now turn to the right image of FIG. 1A (at the time of T2 that follows T1), it may be seen: at the upper part of the right image, due to the motion of the heart, the mitral valve 12 is caused to deviate from the scanning line 11; at the lower part of the right image, also due to the motion of the heart, the myocardium is made at the time of T2 deviate as a whole from the myocardium position at the time of T1. The above deviation will cause image degradation in the final ultrasound image, referring to the lower part of the image in the middle of FIG. 1B, which shows an A-mode image generated from a heart valve that deviates from the scanning line, wherein compared with the clear valve pulsation image at the lower part of the left image, the valve pulsation curve at the lower part of the image in the middle of FIG. 1B has a severe distortion; while the lower part of the right image of FIG. 1B shows a B-mode motion velocity profile, in which a lack of accurately positioning due to the movement of the myocardium causes the myocardium velocity profile to miss a lot of data. And the healthcare providers cannot obtain effective information about the patient's region to be examined from the above images at the lower part of the middle image and at the lower part of the right image of FIG. 1B.

BRIEF DESCRIPTION OF THE INVENTION

The idea of the present invention is to provide an ultrasound imaging method, which can overcome the above image degradation/distortion problems caused by a motion of an organ in the prior art, and can obtain ultrasound image information of an organ to be imaged accurately and clearly.

As one aspect of the present invention, an ultrasound imaging method is provided, comprising the following steps:
(i) selecting an initial ultrasound image frame;
(ii) marking a plurality of marking points on the initial ultrasound image frame;
(iii) tracking updated positions of the plurality of marking points in subsequent ultrasound image frames;
(iv) displaying updated marking points at the updated positions.

As a preference, the following steps may further be comprised: marking a plurality of new marking points in any ultrasound image frame; continuing to implement the steps (iii) and (iv).

As a preference, the following steps may further be comprised: for each frame of ultrasound image:
(v) selecting a first region with a range larger than each marking point around the each marking point;
(vi) selecting a second region with a range larger than the first region around the first region;
(vii) tracking the marking point within a range of a second region in the following frame of ultrasound image, and repeating the steps (iv), (v), (vi) and (vii) when the marking point is tracked down.

As a preference, the following steps may further be comprised: marking two marking points on the initial ultrasound image frame, and determining an M line of an organ by the two marking points, wherein the M line passes through the organ.

As a preference, the following steps may further be comprised: determining updated M lines according to new marking points in the subsequent ultrasound image frames; determining positions of the organ according to the updated M lines, and acquiring an A-mode ultrasound image associated with a motion of the organ according to the positions of the organ in the plurality of image frames.

As a preference, the following steps may further be comprised: marking a plurality of time lines corresponding to the positions of the organ in the plurality of image frames in the A-mode ultrasound image, the plurality of time lines being perpendicular to a time axis of the A-mode ultrasound image.

As a preference, the following steps may further be comprised: the ultrasound imaging method being used for echocardiography, marking more than two marking points along a position of a myocardium on the initial ultrasound image frame.

As a preference, the following steps may further be comprised: determining an updated position and motion of the myocardium according to updated marking points in the subsequent ultrasound image frames; acquiring a B-mode ultrasound image associated with a motion of the myocardium according to the obtained positions and motions of the myocardium in the plurality of image frames.

As a preference, the following steps may further be comprised: marking a plurality of time lines corresponding to the positions of the myocardium in the plurality of image frames in the B-mode ultrasound image, the plurality of time lines being perpendicular to a time axis of the B-mode ultrasound image.

As a preference, the B-mode ultrasound image comprises an ultrasound Doppler imaging.

As a preference, the ultrasound imaging method is AMM or TVM.

As another aspect of the present invention, a non-transient storage medium is provided, which comprises a set of instructions configured to implement on a set of ultrasound images:
(i) selecting an initial ultrasound image frame;
(ii) marking a plurality of marking points on the initial ultrasound image frame;
(iii) tracking updated positions of the plurality of marking points in subsequent ultrasound image frames;
(iv) displaying updated marking points at the updated positions.

As a preference, the set of instructions are configured to further implement: marking a plurality of new marking points in any ultrasound image frame; continuing to implement the steps (iii) and (iv).

As a preference, the set of instructions are configured to further implement:
(v) selecting a first region with a range larger than each marking point around the each marking point;
(vi) selecting a second region with a range larger than the first region around the first region;
(vii) tracking the marking point within a range of a second region in the following frame of ultrasound image, and repeating the steps (iv), (v), (vi) and (vii) when the marking point is tracked down.

As a preference, the set of instructions are configured to further implement: marking two marking points on the initial ultrasound image frame, and determining an M line of an organ by the two marking points, wherein the M line passes through the organ.

As a preference, the set of instructions are configured to further implement: determining updated M lines according to new marking points in the subsequent ultrasound image frames; determining positions of the organ according to the updated M lines, and acquiring an A-mode ultrasound image associated with a motion of the organ according to the positions of the organ in the plurality of image frames.

As a preference, the set of instructions are configured to further implement: marking a plurality of time lines corresponding to the positions of the organ in the plurality of image frames in the A-mode ultrasound image, the plurality of time lines being perpendicular to a time axis of the A-mode ultrasound image.

As a preference, the set of ultrasound images is a set of echocardiographic images, and the set of instructions are configured to further implement: marking more than two marking points along a position of a myocardium on the initial ultrasound image frame.

As a preference, the set of instructions are configured to further implement: determining an updated position and motion of the myocardium according to updated marking points in the subsequent ultrasound image frames; acquiring a B-mode ultrasound image associated with a motion of the myocardium according to the obtained positions and motions of the myocardium in the plurality of image frames.

As a preference, the set of instructions are configured to further implement: marking a plurality of time lines corresponding to the positions of the myocardium in the plurality of image frames in the B-mode ultrasound image, the plurality of time lines being perpendicular to a time axis of the B-mode ultrasound image.

Other features and aspects will be apparent through the following detailed description, figures and claims.

### BRIEF DESCRIPTION OF THE DRAWINGS

The present invention can be better understood in light of the description of exemplary embodiments of the present invention with reference to the accompanying drawings, in which:

FIG. 1A illustrates schematic views of an AMM and a TVM with the organ missing in the prior art;

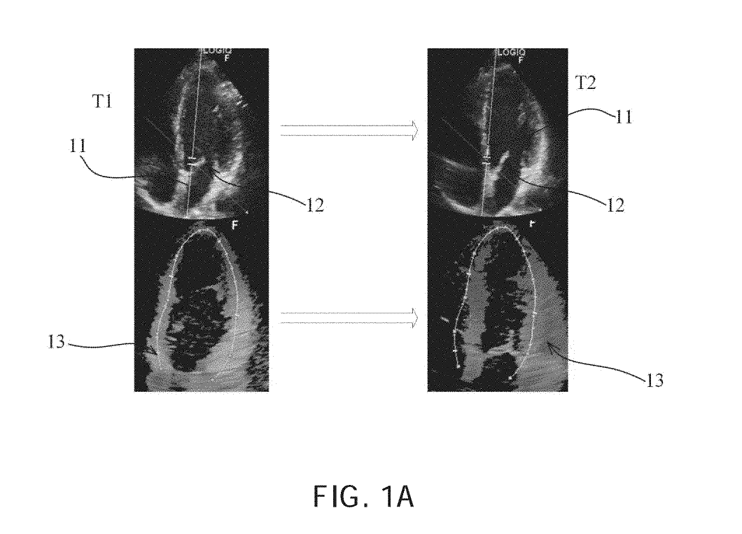

FIG. 1B illustrates views of a normal M-mode imaging, an AMM imaging and a TVM imaging with the organ missing in the prior art;

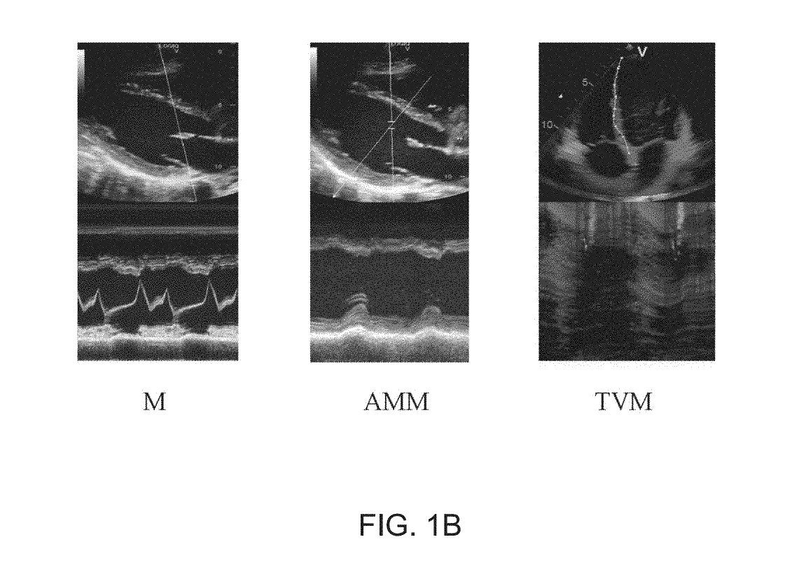

FIG. 2 are schematic views of marking points on a mitral valve and a marking method provided by an embodiment of the present invention;

FIG. 3 are schematic views of tracking the marking points on the mitral valve provided by an embodiment of the present invention;

FIG. 4 are schematic views of M lines and time lines for the mitral valve provided by an embodiment of the present invention;

FIG. 5 are schematic views of marking points on a myocardium and a marking method provided by an embodiment of the present invention;

FIG. 6 are schematic views of tracking the marking points on the myocardium provided by an embodiment of the present invention;

FIG. 7 are schematic views of time lines for the myocardium provided by an embodiment of the present invention;

FIG. 8 shows a method for tracking marking points provided by an embodiment of the present invention;

FIG. 9 is a flow chart of a method provided by an embodiment of the present invention;

FIG. 10 is a comparison view between the prior art and a technical solution of an embodiment of the present invention.

### DETAILED DESCRIPTION OF THE INVENTION

Hereafter, a detailed description will be given for preferred embodiments of the present disclosure. It should be pointed out that in the detailed description of the embodiments, for simplicity and conciseness, it is impossible for the Description to describe all the features of the practical embodiments in details. It should be understood that in the process of a practical implementation of any embodiment, just as in the process of an engineering project or a designing project, in order to achieve a specific goal of the developer and in order to satisfy some system-related or business-related constraints, a variety of decisions will usually be made, which will also be varied from one embodiment to another. In addition, it can also be understood that although the effort made in such developing process may be complex and time-consuming, some variations such as design, manufacture and production on the basis of the technical contents disclosed in the disclosure are just customary technical means in the art for one of ordinary skilled in the art associated with the contents disclosed in the present disclosure, which should not be regarded as insufficient disclosure of the present disclosure.

Unless defined otherwise, all the technical or scientific terms used in the Claims and the Description should have the same meanings as commonly understood by one of ordinary skilled in the art to which the present disclosure belongs. The terms “first”, “second” and the like in the Description and the Claims of the present utility model do not mean any sequential order, number or importance, but are only used for distinguishing different components. The terms “a”, “an” and the like do not denote a limitation of quantity, but denote the existence of a plurality of. The terms “comprises”, “comprising”, “includes”, “including” and the like mean that the element or object in front of the “comprises”, “comprising”, “includes” and “including” covers the elements or objects and their equivalents illustrated following the “comprises”, “comprising”, “includes” and “including”, but do not exclude other elements or objects. The term “coupled” or “connected” or the like is not limited to being connected physically or mechanically, nor limited to being connected directly or indirectly.

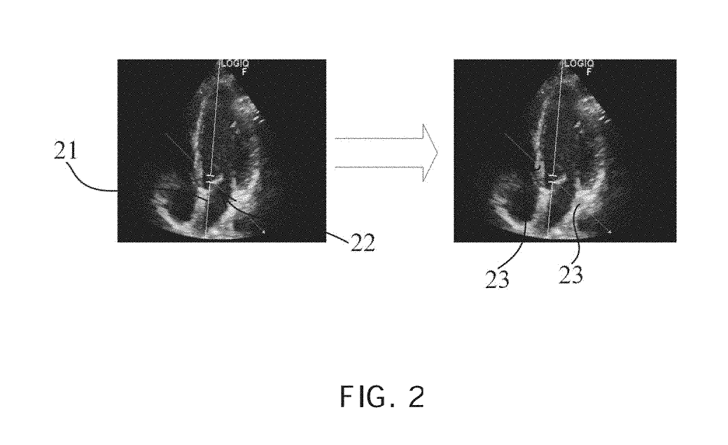

FIG. 2 illustrates schematic views of marking points on a mitral valve and a marking method provided by an embodiment of the present invention, in which an AMM imaging mode is utilized, and during a cine-loop, a frame of image is selected as an initial ultrasound image frame as shown in the left image of FIG. 2, in which a scanning line 21 passes through a mitral valve 22 of a heart; then operation is performed on the initial ultrasound image frame (turn to the right image of FIG. 2), in which the operator selects two positions for marking down two marking points 23 with a connection line there between passing through the mitral valve 22.

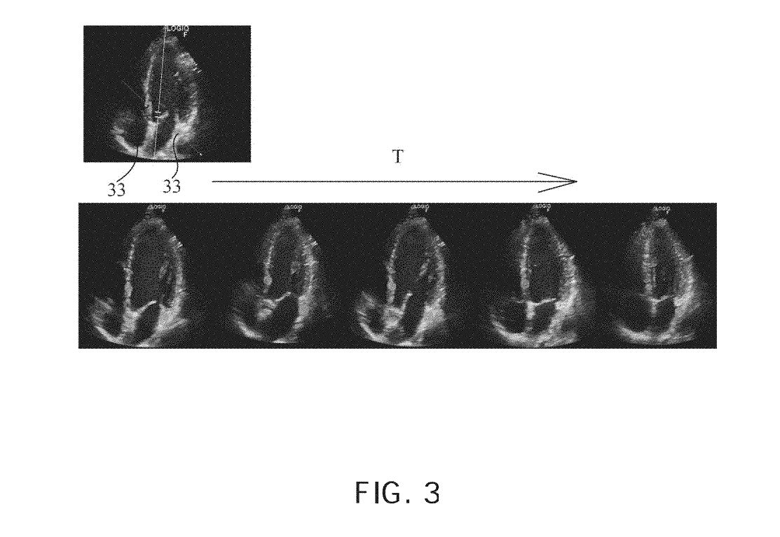

FIG. 3 illustrates an evolution of the ultrasound image frame along the time axis after the marking points have been marked down on the initial ultrasound image frame; the upper image of FIG. 3 is just the initial ultrasound image frame in FIG. 2 with the marking points having been marked thereon, a plurality of images in the lower half part of FIG. 3 are ultrasound images during a period of time subsequent to an initial time corresponding to a time of being the initial ultrasound image frame, and as well known in the art, a mitral valve of a heart (and the heart) is always in a motion status, the position of the mitral valve and its own shape are changing with time, the plurality of ultrasound images at the lower part of FIG. 3 show positions that are constantly changing after the position of the organ corresponding to the initial marking points (two) has moved. And by tracking the positions of the two initial marking points, the mitral valve located at a middle position of the connection line of the two marking points may be accurately positioned.

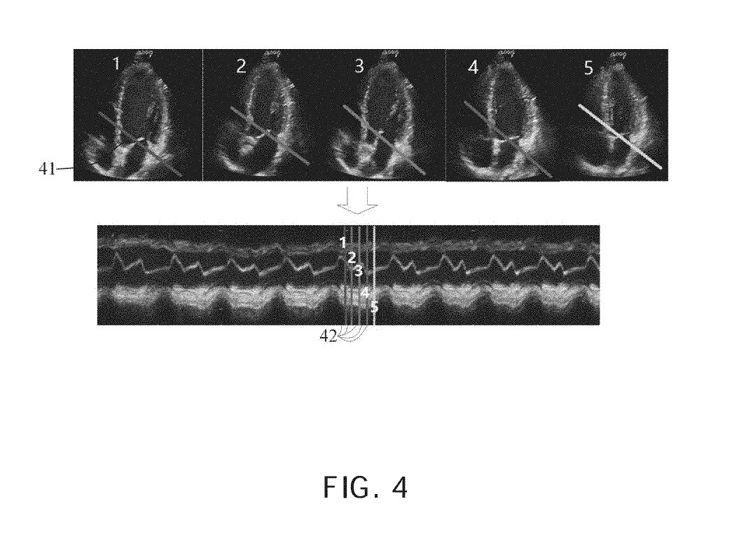

Turn to FIG. 4, the upper part of FIG. 4 (marked with 1-5) more clearly shows the position change of the initial marking points and the connection line between the two initial marking points as the time increases. It can be seen that for any one frame of ultrasound image, the connection lines 41 (referred to as M line for abbreviation) between the two marking points after being marked all pass through the mitral valve, thus the position of the mitral valve may be accurately located. The lower part of FIG. 4 illustrates an A-mode ultrasound image of the mitral valve corresponding to the upper part of FIG. 4, in which the periodic pulsation shows a motion rhythm of the mitral valve. And in the A-mode ultrasound image at the lower part of FIG. 4, five time lines 42 corresponding to the five frames of ultrasound images one by one at the upper part of FIG. 4 are marked. The meaning of the time line is: for example, for the time line 1, its corresponding time is equal to the time of the first frame of ultrasound image at the upper part of FIG. 4, the same for the remaining time lines 2-5, and by comparing the positions of the time lines in the A-mode ultrasound image at the lower part of FIG. 4 and the pulsation values of the positions, and by referring to the shapes of the mitral valve at the corresponding times at the upper part of FIG. 4, a doctor may clearly and intuitively know the shapes, the motions of the mitral valve and the pulsation values generated by the motions thereof at different times, thus it is beneficial to make an accurate condition evaluation for a patient.

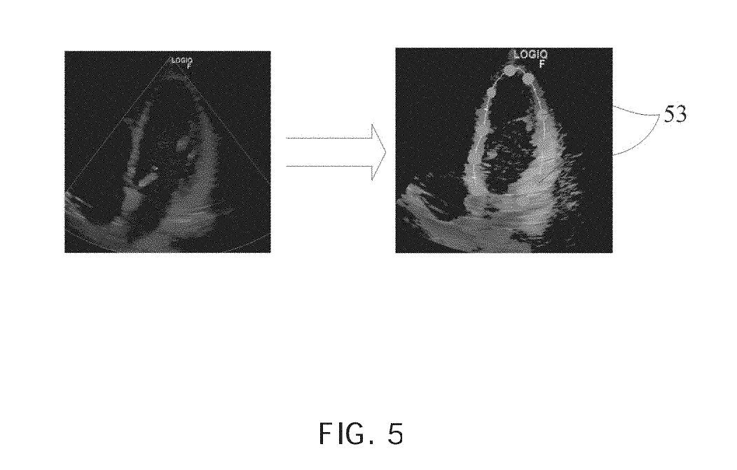

FIG. 5 illustrates schematic views of marking points on a myocardium and a marking method provided by an embodiment of the present invention, in which a TVM imaging mode is utilized, and during a cine-loop, a frame of image is selected as an initial ultrasound image frame as shown in the left image of FIG. 5. Then operation is performed on the initial ultrasound image frame (turn to the right image of FIG. 5), in which the operator selects ten positions for marking down ten marking points 53, which are selected roughly surrounding the myocardium and roughly define the shape of the myocardium.

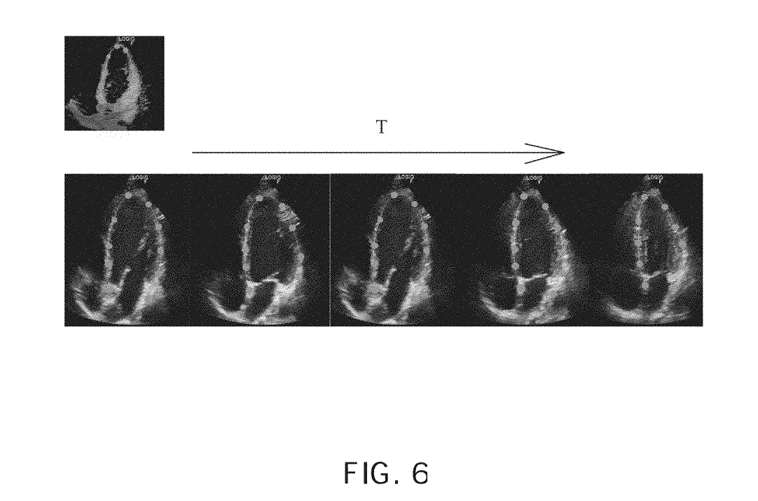

FIG. 6 illustrates an evolution of the ultrasound image frame along the time axis after the marking points have been marked down on the initial ultrasound image frame; the upper image of FIG. 6 is just the initial ultrasound image frame in FIG. 2 with the marking points having been marked thereon, a plurality of images in the lower half part of FIG. 6 are ultrasound images during a period of time subsequent to an initial time corresponding to a time of being the initial ultrasound image frame, and as well known in the art, a myocardium is always in a motion status, the position of the myocardium and its own shape are changing with time, the plurality of ultrasound images at the lower part of FIG. 6 show positions that are constantly changing after the position of the myocardium corresponding to the initial marking points (ten) has moved. And by tracking the positions of the ten initial marking points, the myocardium defined by the ten marking points may be accurately positioned.

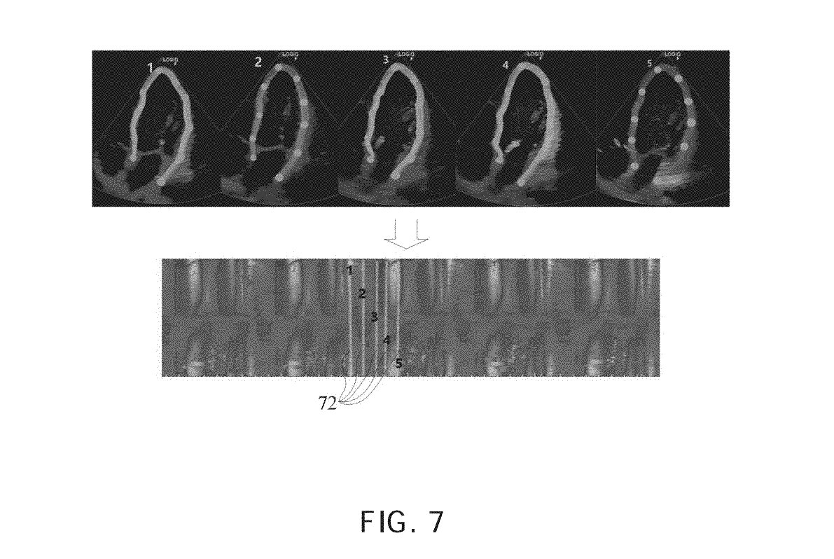

Now turn to FIG. 7, the upper part of FIG. 7 (marked with 1-5) more clearly shows the position change of the ten initial marking points as the time increases. It can be seen that for any one frame of ultrasound image, the position of the myocardium is accurately defined by ten marking points, thus the position of the myocardium may be accurately located. The lower part of FIG. 7 illustrates a B-mode ultrasound image of the myocardium corresponding to the upper part of FIG. 7, and a Doppler imaging mode has been adopted. The red sections represent that the motion of the myocardium is far away from the scanning direction, and the blue sections represent that the motion of the myocardium is towards the scanning direction. By observing the red and blue sections of the myocardium, a doctor may know the motion modes of different regions of the myocardium. In the B-mode Doppler ultrasound image at the lower part of FIG. 7, five time lines 72 corresponding to the five frames of ultrasound images one by one at the upper part of FIG. 7 are marked. The meaning of the time line is: for example, for the time line 1, its corresponding time is equal to the time of the first frame of ultrasound image at the upper part of FIG. 7, the same for the remaining time lines 2-5, and by comparing the positions of the time lines in the B-mode Doppler ultrasound image at the lower part of FIG. 7 and the pulsation values of the positions, and by referring to the shapes of the myocardium at the corresponding times at the upper part of FIG. 7, the doctor may clearly and intuitively know the shapes, the motions and the motion rhythms of the myocardium at different times, thus it is beneficial to make an accurate condition evaluation for a patient.

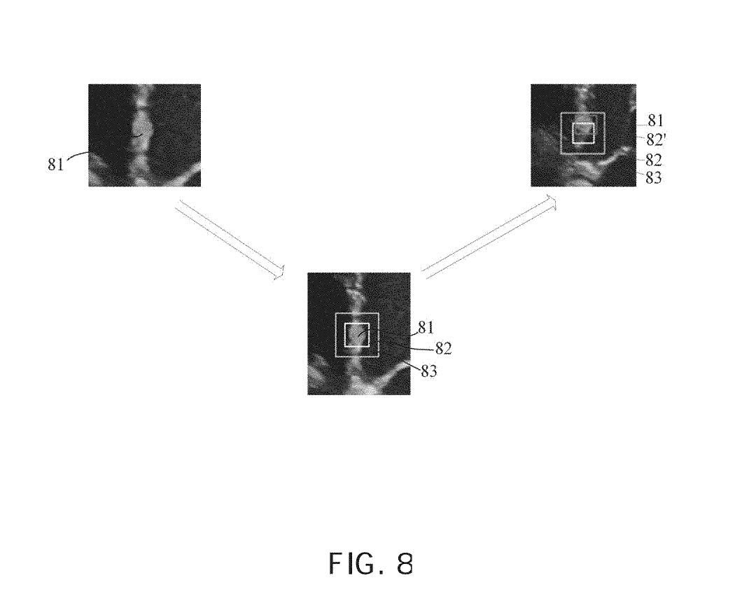

FIG. 8 shows a method for tracking marking points adopted in the embodiments of the present invention, and at first, a marking point 81 is marked on the initial ultrasound image frame (time of T1) as shown in the left image of FIG. 8; then turn to the image frame in the middle of FIG. 8, the time (time of T1) of the image frame is still equal to the time of the left image frame, and a first region 82 with a range larger than each marking point is selected around the each marking point, and a second region 83 with a range larger than the first region 82 is selected around the first region, as shown in the figure, the first region can surround the marking point 81 entirely and the second region 83 can surround the first region 82 entirely; then turn to the right image frame of FIG. 8, whose time (T2) follows the time of T1, which shows an image frame at a time subsequent to the initial image frame and tracking for the marking point, in which the marking point is tracked within the range of the second region of the image frame (marked down within the image frame at the time of T1), and the updated marking point is displayed when the marking point is tracked down. As shown in the figure, it can be seen that from the time of T1 to the time of T2, the marking point has moved, changing from being located at a central part of the second region at the time of T1 to being located at a part above the central part of the second region at the time of T2. And when the new marking point is determined, the positions of the first region and the second region are updated, and then the process consistent with the above process is performed for the subsequent ultrasound image frames (after the time of T2) to continuously track the marking point.

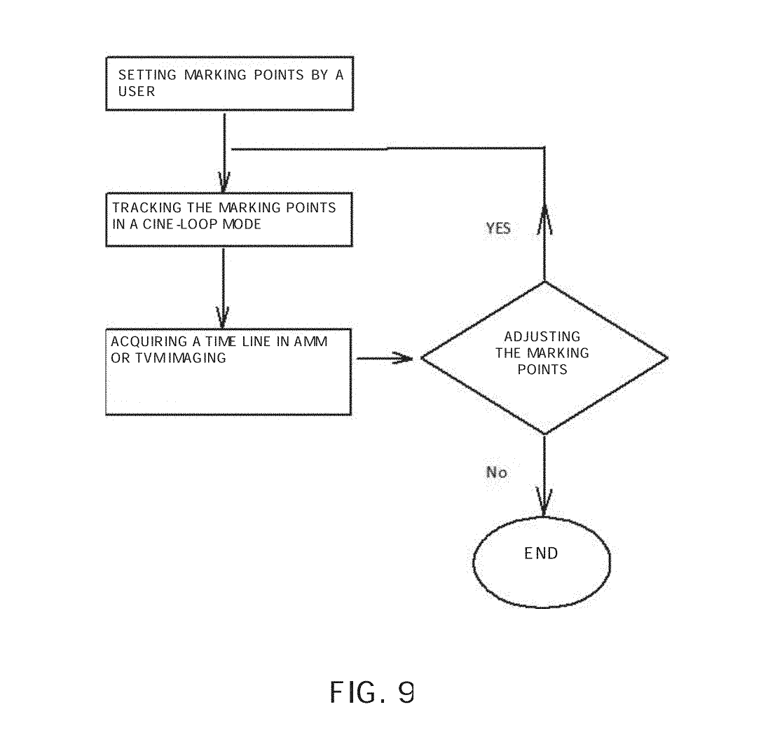

In some embodiments, if the user is not satisfied with the selection for the position of the marking point during tracking, or not satisfied with the selection for the initial ultrasound image frame, a new initial ultrasound image frame can be re-selected or a new marking point can be re-chosen. FIG. 9 illustrates a flow chart for the whole operation. By re-selecting the initial ultrasound image frame and the marking point, the present invention may facilitate the user's accurate positioning for an organ as much as possible, and may clearly obtain the ultrasound image information of an organ to be imaged.

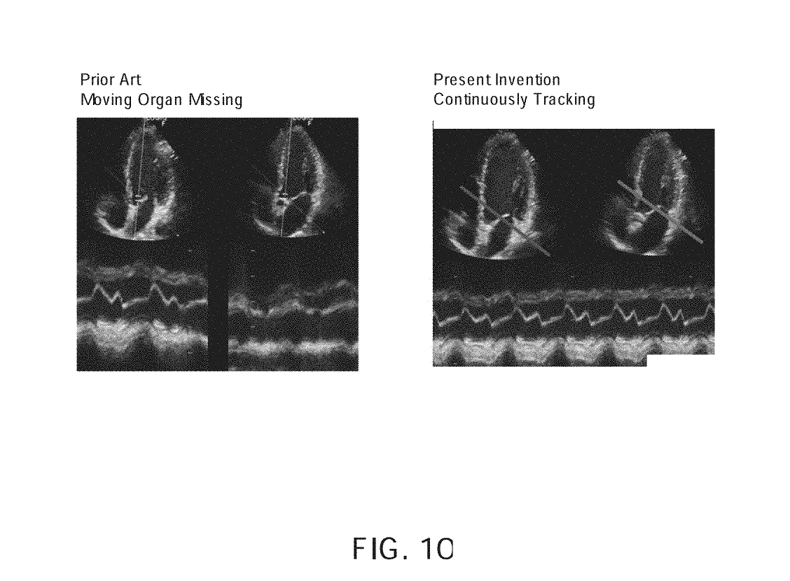

In various embodiments of the present invention, during ultrasound imaging, the position of the moving organ can be accurately positioned, and thus more clear ultrasound image with no distortion can be obtained. As shown in FIG. 10, a comparison view between the prior art and the present invention is illustrated, in which the left image of FIG. 10 shows organ missing due to the motion of the organ in the prior art and thus distortion of the pulsation signal of the organ in the A-mode ultrasound image below; while the right image of FIG. 10 shows the technical solution of the present invention, in which the pulsation signal of the organ in the A-mode ultrasound image shown below is always accurate with no distortion because the technical solution of the present invention can position the moving organ continuously and accurately.

The person skilled in the art may achieve the specific methods in the above various embodiments by utilizing common computer programs which may comprise a set of instructions stored in a non-transient memory. The set of instructions are configured to implement on the image the specific steps in the above various embodiments, and may achieve the above methods by being combined with firmware (for example, display, keyboard) and the like. As used herein, the set of instructions may comprise various commands, which instruct a computer as a processor or a processor to implement specific operations, for example, the methods and processes of the various embodiments of the present invention. The set of instructions may be in a form of software, which can form a part of one or more tangible non-transient computer-readable mediums. The software may be in various forms, e.g., a system software or application software. Moreover, the software may be in forms of single program or module, program module within larger program or a part of program module. The software may also comprise module programming in a form of object-oriented programming The processing on the input data by the processor may respond to the operator's commands, or respond to the previously processed result, or respond to a request made by another processor.

As used herein, the terms “software” and “firmware” may comprise any computer programs stored in a memory for implementation by the computer. The memory comprises RAM memory, ROM memory, EPROM memory, EEPROM memory and non-volatile RAM (NVRAM) memory. The memory types as mentioned above are only exemplary, and thus the memory types that can be used for storing computer programs are not limited.

In addition to any modification as mentioned before, the person skilled in the art can think of many other variations and alternative settings without departing from the spirit and scope of the description, and the attached claims are intended to cover such modifications and settings. Thus, although the above statements have specifically described information in details together with the aspects that are deemed at present as the most practical and preferred, it will be apparent for the common skilled in the art that many modifications (which include but are not limited to forms, functions, operation manners and usages) can be made without departing from the principle and concept stated herein. Furthermore, as used herein, the examples and embodiments are only intended for illustration for all aspects, which should not be interpreted as restriction in any way.

We claim:

1. An ultrasound imaging method, comprising:
(i) selecting an initial ultrasound image frame;
(ii) marking a plurality of marking points on the initial ultrasound image frame;
(iii) tracking updated positions of the plurality of marking points in subsequent ultrasound image frames; (iv) displaying updated marking points at the updated positions.

2. The ultrasound imaging method according to claim 1, further comprising:
marking a plurality of new marking points in any ultrasound image frame;
continuing to implement the steps (iii) and (iv).

3. The ultrasound imaging method according to claim 1, further comprising:
for each frame of ultrasound image:
(v) selecting a first region with a range larger than each marking point around the each marking point;
(vi) selecting a second region with a range larger than the first region around the first region;
(vii) tracking the marking point within a range of a second region in the following frame of ultrasound image, and repeating the steps (iv), (v), (vi) and (vii) when the marking point is tracked down.

4. The ultrasound imaging method according to claims 1, comprising:
marking two marking points on the initial ultrasound image frame, and determining an M line of an organ by the two marking points, wherein the M line passes through the organ.

5. The ultrasound imaging method according to claim 4, wherein updated M lines are determined according to new marking points in the subsequent ultrasound image frames;
positions of the organ are determined according to the updated M lines, and an A-mode ultrasound image associated with a motion of the organ is acquired according to the positions of the organ in the plurality of image frames.

6. The ultrasound imaging method according to claim 5, wherein a plurality of time lines corresponding to the positions of the organ in the plurality of image frames are marked in the A-mode ultrasound image, the plurality of time lines being perpendicular to a time axis of the A-mode ultrasound image.

7. The ultrasound imaging method according to claims 1, wherein the ultrasound imaging method is used for echocardiography, and more than two marking points are marked along a position of a myocardium on the initial ultrasound image frame.

8. The ultrasound imaging method according to claim 7, wherein an updated position and motion of the myocardium is determined according to updated marking points in the subsequent ultrasound image frames; a B-mode ultrasound image associated with a motion of the myocardium is acquired according to the obtained positions and motions of the myocardium in the plurality of image frames.

9. The ultrasound imaging method according to claim 8, wherein a plurality of time lines corresponding to the positions of the myocardium in the plurality of image frames are marked in the B-mode ultrasound image, the plurality of time lines being perpendicular to a time axis of the B-mode ultrasound image.

10. The ultrasound imaging method according to claim 9, wherein the B-mode ultrasound image comprises an ultrasound Doppler imaging.

11. The ultrasound imaging method according to claims 1, wherein the ultrasound imaging method is AMM or TVM.

12. A non-transient storage medium comprising a set of instructions configured to implement on a set of ultrasound images:
(i) selecting an initial ultrasound image frame;
(ii) marking a plurality of marking points on the initial ultrasound image frame;
(iii) tracking updated positions of the plurality of marking points in subsequent ultrasound image frames;
(iv) displaying updated marking points at the updated positions.

13. The non-transient storage medium according to claim 12, wherein the set of instructions are configured to implement:
marking a plurality of new marking points in any ultrasound image frame;
continuing to implement the steps (iii) and (iv).

14. The non-transient storage medium according to claim 12, wherein the set of instructions are configured to implement:
(v) selecting a first region with a range larger than each marking point around the each marking point;
(vi) selecting a second region with a range larger than the first region around the first region;
(vii) tracking the marking point within a range of a second region in the following frame of ultrasound image, and repeating the steps (iv), (v), (vi) and (vii) when the marking point is tracked down.

15. The non-transient storage medium according to claims 12, wherein the set of instructions are configured to implement:
marking two marking points on the initial ultrasound image frame, and determining an M line of an organ by the two marking points, wherein the M line passes through the organ.

16. The non-transient storage medium according to claim 15, wherein the set of instructions are configured to implement: updated M lines are determined according to new marking points in the subsequent ultrasound image frames; positions of the organ are determined according to the updated M lines, and an A-mode ultrasound image associated with a motion of the organ is acquired according to the positions of the organ in the plurality of image frames.

17. The non-transient storage medium according to claim 16, wherein the set of instructions are configured to implement: marking a plurality of time lines corresponding to the positions of the organ in the plurality of image frames in the A-mode ultrasound image, the plurality of time lines being perpendicular to a time axis of the A-mode ultrasound image.

18. The non-transient storage medium according to claims 12, wherein the set of ultrasound images is a set of echocardiographic images, and the set of instructions are configured to implement:
marking more than two marking points along a position of a myocardium on the initial ultrasound image frame.

19. The non-transient storage medium according to claim 18, wherein the set of instructions are configured to implement: determining an updated position and motion of the myocardium according to updated marking points in the subsequent ultrasound image frames; acquiring a B-mode ultrasound image associated with a motion of the myocardium according to the obtained positions and motions of the myocardium in the plurality of image frames.

20. The non-transient storage medium according to claim 19, wherein the set of instructions are configured to implement: marking a plurality of time lines corresponding to the positions of the myocardium in the plurality of image frames in the B-mode ultrasound image, the plurality of time lines being perpendicular to a time axis of the B-mode ultrasound image.
